(19) United States
(12) Patent Application Publication
(10) Pub. No.: US 2014/0112954 A1
(43) Pub. Date: Apr. 24, 2014

(54) METHOD FOR PREPARING HBV VACCINE COMPRISING ALUMINUM ADJUVANT

(71) Applicants: Guixing Li, Dalian (CN); Ping Zhang, Dalian (CN); Haichun Zhang, Dalian (CN); Xinting Chen, Dalian (CN); Xiaoyu Li, Dalian (CN); Juan Zhang, Dalian (CN); Caixia Su, Dalian (CN)

(72) Inventors: Guixing Li, Dalian (CN); Ping Zhang, Dalian (CN); Haichun Zhang, Dalian (CN); Xinting Chen, Dalian (CN); Xiaoyu Li, Dalian (CN); Juan Zhang, Dalian (CN); Caixia Su, Dalian (CN)

(73) Assignee: DALIAN HISSEN BIO-PHARM CO., LTD., Dalian, Liaoning (CN)

(21) Appl. No.: 14/117,937

(22) PCT Filed: Apr. 18, 2012
(86) PCT No.: PCT/CN2012/074274

§ 371 Date: Jan. 10, 2014

(30) Foreign Application Priority Data
May 16, 2011 (CN) 201110126060.4

(51) Int. Cl. A61K 39/29

(52) U.S. Cl. CPC A61K 39/292
USPC 424/227.1; 435/69.3

(57) ABSTRACT

The present invention discloses a method for preparing HBV Vaccine comprising aluminum adjuvant, which belongs to biological technology field. The method, which is characterized in that aluminum adjuvant Al(OH)₃ is produced by an on-line reaction, comprises mixing PBS buffer solution and potassium aluminum sulfate (KAl(SO4)₂) solution with hepatitis B surface antigen stock solution, adding sodium hydroxide (NaOH) solution into the mixed solution, so that the adjuvant is continuously produced and hepatitis B surface antigens are continuously coated and absorbed simultaneously. The process is called “in-situ adsorption”.

### TECHNICAL FIELD

The present invention relates to a method for preparing a vaccine, especially a method for preparing a hepatitis B vaccine comprising aluminum adjuvant. It belongs to the biotechnical field.

### BACKGROUND OF THE INVENTION

At present, about 350 million people around the world are suffering from Hepatitis B virus (HBV); in China, 130 million people are suffering from chronic HBV which is seriously threatening the health of mankind; sustainable infection will lead to chronic hepatitis, liver cirrhosis and liver cancer. The medical circle has no efficient drug to root out hepatitis; large-scale vaccination of hepatitis B vaccine is the most economical and effective method to prevent HBV; it is also deemed as the fundamental method to reduce HBV hazard.

Hepatitis B vaccine mainly consists of hepatitis B surface antigen; the adjuvant comprises the traditional aluminum hydroxide.

In 1926, Glenny first discovered diphtheria toxoid (DT) suspension sedimented by aluminum adjuvant enjoys higher antigenicity than toxoid; therefore, various adjuvants have become the research focus. The existing materials have revealed that hepatitis B vaccine with new adjuvant includes: hepatitis B vaccine with adjuvant system coordinated between lipid A and aluminum salt, hepatitis B vaccine with nano adjuvant, hepatitis B vaccine with lipid, hepatitis B vaccine with Granulocyte Macrophage-Colony Stimulating Factor (Gm-CSF) adjuvant, hepatitis B vaccine with plant adjuvant, hepatitis B vaccine with immunostimulatory sequence adjuvant and hepatitis B vaccine with microsphere delivery adjuvant system (“Progress in the developments of hepatitis B vaccine and its adjuvants” edited by Feng Li, Zhao Huan, Xue Shike and Qi Xianrong, Chinese journal of New Drugs, 2007, vol 16, No. 20)). However, hepatitis B vaccine with new adjuvant has failed to launch out the mass production; instead, it is still in the research phase. Mass production requires for further investigation and verification. Until now, aluminum salt adjuvant is deemed as the vaccination adjuvant for mankind as approved by Food and Drug Administration; upon long-term application, the effectiveness and safety of aluminum hydroxide adjuvant have been verified by practice. Therefore, it is highly necessary to optimize the widely applied aluminum hydroxide adjuvant, realize further optimization and upgrading based on existing effectiveness and safety and upgrade the immunogenicity of hepatitis B vaccine.

According to recent statistics of WHO, about 5% people with three hepatitis B vaccines in 0, 1^{st} and 6^{th} month prove to be invalid. Gupta has proven that vaccination effect of absorbed vaccine of aluminum adjuvant depends on the adjuvant absorption of antigen; the absorption rate of antigen and adjuvant is deemed as an important factor in determining the vaccination effect: higher absorption rate leads to better vaccination effect; lower absorption rate leads to worse vaccination effect (Gupta R k, Rost B E, Relyveld E, et al. Vaccine design: the subunit and adjuvant approach. NEW York: Plenum Press, 1995:229-248; Liu Kaiyun, Zou Quanming “Application of aluminum adjuvant in the developments helicobacter pylori vaccine”, Immunological Journal, 2004, 20 (3): S50). WHO requires an absorption rate of at least 80% in diphtheria and tetanus toxoid.

The normal vaccine with aluminum adjuvant comprises two categories: aluminum sedimentation vaccine and aluminum absorption vaccine. The aluminum sedimentation vaccine aims to add aluminum adjuvant suspension into the antigen; the aluminum absorption vaccine aims to add antigen solution into the aluminum hydroxide or phosphoric acid aluminum adjuvant. The existing aluminum absorption process of hepatitis B vaccine aims to first prepare Al(OH)₃ adjuvant, wherein AlCl₃ solution and NaOH solution have a chemical reaction to produce 1 mg/ml Al(OH)₃; then add antigen to the hepatitis B surface and thus prepare hepatitis B vaccine. The existing aluminum adjuvant absorption process suffers from such problems as low adjuvant absorption rate, high vaccination volume and poor seroconversion transfer rate.

### SUMMARY OF THE INVENTION

The invention aims to provide the methods of for preparation a hepatitis B vaccine containing aluminum adjuvant. In the existing aluminum absorption process of hepatitis B vaccine, the Al(OH)₃ adjuvant was prepared firstly, that is, 1 mg/ml Al(OH)₃ was produced by reaction of AlCl₃ solution and NaOH solution, then hepatitis B surface antigen was added to the reaction solution, thus hepatitis B vaccine was prepared. The preparation process is as follows: adopt injection water to prepare 10% AlCl₃ solution, 0.5 mol/L NaOH solution and 0.9% NaCl solution; mix up, add 10% AlCl₃ solution into the flask according to the volume with an ultimate concentration of 1.0 mg/ml in aluminum adjuvant and then add 0.5 mol/L NaOH solution, stop adding solution once pH value is 7.0; supplementing 0.9% NaCl until the needed volume; implement moist heat sterilization under 121° C. for 30 minutes and prepare the aluminum adjuvant. Take the stock solution of hepatitis B surface antigen according to the volume with an ultimate antigen concentration of 20 μg/ml in the semi-finished product, and add the aluminum adjuvant with equivalent volume, mixing adequately and obtaining the semi-finished product of hepatitis B vaccine.

Compared with the prior art, the invention has the advantages that: Al(OH)₃ adjuvant is not prepared in the preliminary phase; instead, it mixes up PBS buffer solution, KAl(SO₄)₂ solution and hepatitis B surface antigen stock solution, adds NaOH solution to the mixed solution, the Al(OH)₃ adjuvant is produce by an on-line reaction, so that the Al(OH)₃ adjuvant is continuously produced and hepatitis B surface antigens are continuously coated and absorbed. The process is called “In-situ absorption”. The prepared sample refers to milky white gel semisolid substance; it is able to strongly absorb protein antigen in the solution and form sediment. It will form an “antigen warehouse” upon vaccination into the body and slowly release the antigen, therefore, it has adequately lengthened the effect time of antigen and played the role of long-term protection. At the same time, it is able to promote response of partial (injection position) macrophage. The technical plan of this invention is as follows:

A method for preparation of a hepatitis B vaccine comprising aluminum adjuvant, characterized in that aluminum adjuvant Al(OH)₃ was produced by an on-line reaction, comprises mixing PBS buffer solution, KAl(SO4)₂ solution and hepatitis B surface antigen stock solution, adding NaOH solution into the mixed solution, so that the Al(OH)₃ adjuvant is continuously produced and hepatitis B surface antigens are continuously coated and absorbed to the adjuvant simultaneously, the method comprises the following steps:

\textcircled{1} adopting injection water to prepare 3 mmol/L PBS buffer solution, 10 wt % KAl(SO₄)₂ solution, 0.5 mol/L NaOH solution and 0.9 wt % NaCl solution, and the prepared solutions were sterilized by filtration with 0.22 μm membrane filter;

\textcircled{2} adding 200 ml PBS to the reaction vessel, under the mixing condition, adding the hepatitis B surface antigen stock solution according to the content with an ultimate antigen concentration of 20 μg/ml in the semi-finished product, then adding 150 ml PBS solution and 10 wt % KAl(SO₄)₂ solution according to the aluminum content with an ultimate concentration of 0.5 mg/ml in the semi-finished product, then adding 0.5 mol/L NaOH solution, adjust pH value to 7.0, finally supplementing the 0.9 wt % NaCl solution until 600 ml and sealing the mixed solution; a semi-finished product of hepatitis B vaccine was obtained via sedimentation and washing process for the mixed solution.

The hepatitis B surface antigen stock solution is prepared according to the normal technologies;

The sedimentation and washing process specified in above step \textcircled{2} is as follows:

i. Sedimenting the sealed mixed solution for 12-19 h, then removing the supernatant and reserve the sediment, adding the 0.9 wt % NaCl solution to the sediment until the original volume of semi-finished product, mixing the material for 30 minutes, sealing and sedimenting for the second time;

ii. Repeating the above step i twice;

iii. After 4^{th} precipitation for 20 h, removing the supernatant, then adding the 0.9 wt % NaCl solution until the original volume of semi-finished product, mixing for 30 minutes and mix up evenly, obtaining the semi-finished product of hepatitis B vaccine.

The hepatitis B surface antigen comprises recombinant yeast hepatitis B surface antigen and recombinant CHO hepatitis B surface antigen.

The hepatitis B surface antigen comprises the recombinant yeast hepatitis B surface antigen.

The recombinant yeast hepatitis B surface antigen comprises recombinant hansenula polymorpha hepatitis B surface antigen.

The stock solution of recombinant hansenula polymorpha hepatitis B surface antigen is prepared by the following process:

I. Collection of fermentation broth: preparing a batch strain of working seed by fourth amplification of national approved original bacteria of recombinant hansenula polymorpha hepatitis B vaccine, the batch strain seeded in 300 ml growth medium, cultured under 30˜35° C. for 24 h, and then the culture medium was transferred to 3 L growth medium further cultured under 30˜35° C. for 24 h; then the medium was transferred to 30 L growth medium cultured under 30˜35° C. for 15 h and the resulting medium ultimately transferring to 200 L growth medium and cultured under 30˜35° C. for 65-70 h in a condition of a dissolved oxygen for above 20%, a pH value at 6.8 and a air flow between 30˜200 ml/h, collecting the fermentation broth of hansenula polymorpha cell expressing the hepatitis B surface antigen;

II. Preliminary purification: Grinding and crushing the fermentation broth of hansenula polymorpha cell by physical crushing method to realize a crushing rate of above 90%, removing the cell debris by centrifugation at 4000 rpm, the supernatant was performed two aqueous phase extraction by treating with NaCl/PEG6000 for 8-10 h and centrifuged with a speed of 6000 rpm, reserving the supernatant, then the supernatant was absorbed with silica gel solution for 10-16 h, and desorpted for 1 h at 60° C., then centrifuged at 4000 rpm, reserving the supernatant;

III. Fine purification: purificating the preliminarily purified sample by anionic column chromatography, washing the column with 100 mmol/L tris buffer solution, monitored at absorbance of 280 nm and collecting protein peak with OD value of above 1, concentrating the protein peak for 10 times by 50K membrane, then implement centrifugal force to the belt of equivalent density area and monitored at absorbance of 280 nm, collecting a protein peak with OD value of above 1 and the protein peak was further purified by molecule sieve column chromatography, the column was eluted with biological buffer solution to perform protein separation and desalination, the elutes monitored at absorbance of 280 nm, collecting HBsAg protein peak with OD value of above 0.8 so that its purity is above 99.0%; ultimately diluting HBsAg protein peak until a content of 100-300 μg/ml, and sterilizing by filtration with 0.22 μm membrane filter, obtaining the stock solution of recombinant hansenula polymorpha hepatitis B surface antigen;

The growth medium comprises the following ingredients: glycerin, magnesium sulfate, potassium chloride, sodium chloride and ammonium hydrogen phosphate, according to the mass ratio in 1 L is 5:2:1:0.1:4, it is prepared by injection water and sterilized under 121° C. for 30 minutes.

The national approved original strain of recombinant hansenula polymorpha hepatitis B vaccine enjoys a strain No. of HBsAgU35-16-9 (Chinese pharmacopoeia, 2010 version, three volumes, recombinant hepatitis B vaccine (hansenula polymorpha yeast), Page 132); it is preserved by Dalian Hanxin Biological Pharmaceutical Co., Ltd.; it can be purchased by commercial channels

The semi-finished product of hepatitis B vaccine prepared by this invention is processed and treated by normal technologies and then prepared into finished hepatitis B vaccine.

The beneficial effect of this invention is as follows: Compared with the traditional process, the solution adopted for this invention (in-situ absorption method) is sterilized by filtration. Therefore, high-pressure sterilization is not needed, so it has simplified the process and reduced the production costs. Al(OH)₃ adjuvant in this invention is prepared by in-situ reaction so that absorption rate of hepatitis B surface antigen is greatly increased, Therefore, immunogenicity of antigen is increased, and the detection result of mouse ED₅₀ is much superior to the former and it is able to even effectively lead to immunoreaction in the body and produce more protective antibodies. Practice has proven that hepatitis B vaccine with aluminum adjuvant produced by this method enjoys such strengths as small vaccination volume, less poor reaction, high-level anti-body response upon immunity and high seroconversion transfer rate.

### DETAILED DESCRIPTION OF THE INVENTION

Specify the invention by examples and comparison examples; however, the invention is not limited by the following examples.

Description of Raw Materials:

Strain: The original strain of recombinant hansenula polymorpha hepatitis B vaccine expressing HBsAg that is constructed with DNA recombinant technique (researched and developed by Dalian Hanxin Biological Pharmaceutical Co., Ltd.) enjoys a strain No. of HBsAgU35-16-9. (Chinese pharmacopoeia, 2010 version, three volumes, recombinant hepatitis B vaccine (hansenula polymorpha yeast), Page 132). It is preserved by Dalian Hanxin Biological Pharmaceutical Co., Ltd.

The fermentation growth medium comprises the following ingredients: glycerin, magnesium sulfate, potassium chloride, sodium chloride and ammonium hydrogen phosphate. The mass ratio in 1 L is 5:2:1:0.1:4. It is prepared by injection water and sterilized under 121° C. for 30 minutes.

Sodium chloride solution: Concentration is 3 mol/L; it is prepared by injection water; it is sterilized by filtration with 0.22 μm membrane filter;

PGE6000 solution: Concentration is 50%; it is prepared by injection water; it is sterilized under 121° C. for 30 minutes;

Silica gel solution: Concentration is 7.5%; it is prepared by injection water; it is sterilized under 121° C. for 30 minutes;

Tris-HCl+2 mol/L NaCl solution: Tris (batch No.: WF0131LA01, USA); concentration is 0.1 mol/L; pH value is 8.5; it is prepared by injection water; it is sterilized by filtration with 0.22 μm membrane filter;

Potassium bromide solution: Density is 1.04 g/ml, 1.28 g/ml, 1.34 g/ml respectively; it is prepared by injection water; it is sterilized by filtration with 0.22 μm membrane filter;

Sodium chloride solution: Concentration is 0.9%; it is prepared by injection water; it is sterilized by filtration with 0.22 μm membrane filter;

Phosphate buffer solution: sodium hydrogen phosphate and monosodium phosphate have a concentration of 3 mmol/L; it is prepared by injection water; it is sterilized by filtration with 0.22 μm membrane filter;

AlCl₃ solution: Concentration is 10%; it is prepared by injection water;

Sodium hydroxide solution: Concentration is 0.5 mol/L; it is prepared by injection water;

Aluminum potassium sulfate solution: Concentration is 10%; it is prepared by injection water; it is sterilized by filtration with 0.22 μm membrane filter;

### Example 1

### Preparation of Concentrate of Recombinant (Hansenula Polymorpha Yeast) Hepatitis B Surface Antigen

Fermentation: one batch strain of working seed of recombinant hansenula polymorpha yeast was prepared (it is obtained by amplification of the original strain of hepatitis B vaccine of recombinant hansenula polymorpha yeast (strain No.: HBsAgU35-16-9)) and the batch strain of working seed was seeded in 300 ml growth medium and cultured for 24 h at 35° C. Then the medium was transferred to 3 L growth medium further cultured for 24 h at 35° C. and the resulting medium was transferred to 30 L growth medium cultured for 15 h at 35° C., Finally, the culture medium was transferred to 200 L growth medium and cultured at 35° C. for 65 h a in a condition of a dissolved oxygen for above 20%, a pH value at 6.8 and a air flow between 30˜200 ml/h, obtained fermentation broth of hansenula polymorpha cell expressing hepatitis B surface antigen for about 240 L;

Preliminary purification: Grinding and crushing the fermentation broth hansenula polymorpha cell by physical crushing method to realize a crushing rate of 92%, removing cell debris by centrifugation at 4000 rpm and the supernatant was collected. The supernatant was performed two aqueous phase extraction by treating with NaCl/PEG6000 for 9 h and centrifuged with a speed of 6000 rpm, collected about 230 L supernatant, and then the supernatant was absorbed by silica gel solution for 15 h and desorpted under 60° C. for 1 h, then centrifuged at a rotational speed of 4000 rpm and collected about 110 L supernatant; fulfill the preliminary purification.

Fine purification: purificating the preliminarily purified sample by DEAE Sepharose FF anionic column chromatography; the sample loading quantity is 10 times of the column volume, after loading the sample to the column, the column was eluted with 100 mmol/L tris buffer solution, velocity flow of 60 L/h. The elutes was monitored at absorbance of 280 nm, and collected the protein peak with OD value of above 1; concentrating the collected protein peak and implemented a centrifugal process to the belt in the equivalent density area; monitored at absorbance of 280 nm and collected the protein peak with OD value of above 1. The collected protein peak was further purified by molecule sieve column chromatography, the column was eluted with 0.9% NaCl solution for protein separation and desalination; the elutes was monitored at absorbance of 280 nm and collecting HBsAg protein peak with OD value of above 0.8 for 28.2 L; adopt HPLC method to inspect the purity of above 99.0%; ultimately dilute HBsAg protein content until 220 μg/ml by phosphate buffer solution; adopt 0.22 μm sterilization filter membrane for sterilization and filtration for 60 L. Therefore, it will obtain the stock solution of recombinant hansenula polymorpha yeast hepatitis B surface antigen.

### Example 2

### Preparation of Semi-Finished Hepatitis B Vaccine (Aluminum Adjuvant+HBsAg Process—Prior art); Batch No. of Semi-Finished Product is S201001

Preparation of aluminum adjuvant: mix up, adding 54 ml of 10% AlCl₃ solution into the flask, then adding 0.5 mol/L NaOH solution at a speed of 50 ml/min and the mixture (reaction solution) was subjected to measure pH value at any time; stop adding solution once pH value is 7.00, and 110 ml of 0.5 mol/L NaOH solution was further added, supplement 436 ml of 0.9% NaCl solution until the ultimate volume of 600 ml, the solution was sterilized by moist heat sterilization under 121° C. for 30 minutes, obtaining the aluminum adjuvant.

Dilution of stock solution: adopting 54.5 ml stock solution with a protein content of 220 μg/ml (prepared in example 1) and put into 500 ml conical flask; adding 300 ml of 0.9% NaCl solution so that protein content is 40 μg/ml.

Absorption of semi-finished product: mix up, 300 ml aluminum adjuvant was added to the other flask, then the 300 ml diluted stock solution was added and mixed for 30 minutes, obtaining the semi-finished product of hepatitis B vaccine; the batch No. of semi-finished product is S201001.

### Example 3

### Preparation of Semi-Finished Hepatitis B Vaccine (Aluminum Adjuvant+HBsAg Process—Prior art); Batch No. of Semi-Finished Product is S201002

Preparation of aluminum adjuvant: mix up, adding 54 ml of 10% AlCl₃ solution into the flask, then adding 0.5 mol/L NaOH solution at a speed of 50 ml/min and the mixture (reaction solution) was subjected to measure pH value at any time; stop adding solution once pH value is 6.95, and 108 ml of 0.5 mol/L NaOH solution was further added, supplement 438 ml of 0.9% NaCl solution until the ultimate volume of 600 ml, the solution was sterilized by moist heat sterilization under 121° C. for 30 minutes, obtaining the aluminum adjuvant.

Dilution of stock solution: adopting 54.5 ml stock solution with a protein content of 220 μg/ml (prepared in example 1) and put into 500 ml conical flask; adding 300 ml of 0.9% NaCl solution so that protein content is 40 μg/ml.

Absorption of semi-finished product: mix up, 300 ml aluminum adjuvant was added to the other flask, then the 300 ml diluted stock solution was added and mixed for 30 minutes, obtaining the semi-finished product of hepatitis B vaccine; the batch No. of semi-finished product is S201002.

### Example 4

### Preparation of Semi-Finished Hepatitis B Vaccine (Aluminum Adjuvant+HBsAg Process—Prior art); Batch No. of Semi-Finished Product is S201003

Preparation of aluminum adjuvant: mix up, adding 54 ml of 10% AlCl₃ solution into the flask, then adding 0.5 mol/L NaOH solution at a speed of 50 ml/min and the mixture (reaction solution) was subjected to measure pH value at any time; stop adding solution once pH value is 7.00, and 115 ml of 0.5 mol/L NaOH solution was further added, supplement 431 ml of 0.9% NaCl solution until the ultimate volume of 600 ml, the solution was sterilized by moist heat sterilization under 121° C. for 30 minutes, obtaining the aluminum adjuvant.

Dilution of stock solution: adopting 54.5 ml stock solution with a protein content of 220 μg/ml (prepared in example 1) and put into 500 ml conical flask, adding 300 ml of 0.9% NaCl solution so that protein content is 40 μg/ml.

Absorption of semi-finished product: mix up, 300 ml aluminum adjuvant was added to the other flask, then the 300 ml diluted stock solution was added and mixed for 30 minutes, obtaining the semi-finished product of hepatitis B vaccine; the batch No. of semi-finished product is S201003.

### Example 5

### Production Process of Semi-Finished Hepatitis B Vaccine (In-Situ Absorption—the Invention); Batch No. of Semi-Finished Product is S201004

Mix up; adding 200 ml PBS solution into the flask; adding 54.5 ml of 220 μg/ml stock solution (prepared in example 1); then adding 150 ml PBS solution and adding 54.7 ml of 10% KAl(SO₄)₂ solution; then adding 0.9% NaCl solution until total weight 520 ml, start adding 0.5 mol/L NaOH solution into the flask at a speed of 50 ml/min; in the addition of 45 ml of 0.5 mol/L NaOH solution, adopt sample and measure pH value; once pH value is 5.50, control the addition speed of 0.5 mol/L NaOH solution at 20 ml/min; adopt sample and measure pH value at any time; stop adding 0.5 mol/L NaOH solution once pH value is 7.00; add 56 ml solution in total; ultimately supplement 0.9% NaCl solution until 600 ml; mix up for 10 minutes and then stop mixing; seal up and sediment.

First washing and sedimentation: Upon sedimentation for 19 h, removing the supernatant, then adding 0.9% NaCl into the flask until 600 ml and it was mixed for 10 minutes, close the mixing function and seal up and sediment. Second washing and sedimentation: Upon sedimentation for 12 h, removing the supernatant, then adding 0.9% NaCl into the flask until 600 ml and it was mixed for 10 minutes, close the mixing function and seal up and sediment. Third washing and sedimentation: Upon sedimentation for 12 h, removed the supernatant, then supplement 0.9% NaCl into the flask until 600 ml and it was mixed for 10 minutes, close the mixing function and seal up and sediment. Preparation of semi-finished products: Upon sedimentation for 20 h, removing the supernatant, then supplement 0.9% NaCl into the flask until 600 ml; mix up evenly for 30 minutes, obtaining the semi-finished hepatitis B vaccine. The batch No. of semi-finished product is S201004.

### Example 6

### Production Process of Semi-Finished Hepatitis B Vaccine (In-Situ Absorption—the Invention); Batch No. of Semi-Finished Product is S201005

Mix up; adding 200 ml PBS solution into the flask; adding 54.5 ml of 220 μg/ml stock solution (prepared in example 1); then adding 150 ml PBS solution and adding 54.7 ml of 10% KAl(SO₄)₂ solution; then adding 0.9% NaCl solution until total weight 520 ml, start adding 0.5 mol/L NaOH solution into the flask at a speed of 50 ml/min; in the addition of 45 ml of 0.5 mol/L NaOH solution, adopt sample and measure pH value; once pH value is 5.5, control the addition speed of 0.5 mol/L NaOH solution at 20 ml/min; adopt sample and measure pH value at any time; stop adding 0.5 mol/L NaOH solution once pH value is 6.88; add 52 ml solution in total; ultimately supplement 0.9% NaCl solution until 600 ml; mix up for 10 minutes and then stop mixing; seal up and sediment.

First washing and sedimentation: Upon sedimentation for 19 h, removing the supernatant, then adding 0.9% NaCl into the flask until 600 ml and it was mixed for 10 minutes, close the mixing function and seal up and sediment. Second washing and sedimentation: Upon sedimentation for 12 h, removing the supernatant, then adding 0.9% NaCl into the flask until 600 ml and it was mixed for 10 minutes, close the mixing function and seal up and sediment. Third washing and sedimentation: Upon sedimentation for 12 h, removing the supernatant, then supplement 0.9% NaCl into the flask until 600 ml and it was mixed for 10 minutes, close the mixing function and seal up and sediment. Preparation of semi-finished products: Upon sedimentation for 20 h, removing the supernatant, then supplemented 0.9% NaCl into the flask until 600 ml; mix up evenly for 30 minutes, obtaining the semi-finished hepatitis B vaccine. The batch No. of semi-finished product is S201005.

### Example 7

### Production Process of Semi-Finished Hepatitis B Vaccine (In-Situ Absorption—the Invention); Batch No. of Semi-Finished Product is S201006

Mix up; adding 200 ml PBS solution into the flask; adding 54.5 ml of 220 μg/ml stock solution (prepared in example 1); then adding 150 ml PBS solution and adding 54.7 ml of 10% KAl(SO₄)₂ solution; then adding 0.9% NaCl solution until total weight 520 ml, start adding 0.5 mol/L NaOH solution into the flask at a speed of 50 ml/min; in the addition of 45 ml of 0.5 mol/L NaOH solution, adopt sample and measure pH value; once pH value is 5.5, control the addition speed of 0.5 mol/L NaOH solution at 20 ml/min; adopt sample and measure pH value at any time; stop adding 0.5 mol/L NaOH solution once pH value is 6.92; add 58 ml solution in total; ultimately supplement 0.9% NaCl solution until 600 ml; mix up for 10 minutes and then stop mixing; seal up and sediment.

First washing and sedimentation: Upon sedimentation for 19 h, removing the supernatant, then adding 0.9% NaCl into the flask until 600 ml and it was mixed for 10 minutes, close the mixing function and seal up and sediment. Second washing and sedimentation: Upon sedimentation for 12 h, removing the supernatant, then added 0.9% NaCl into the flask until 600 ml and it was mixed for 10 minutes, close the mixing function and seal up and sediment. Third washing and sedimentation: Upon sedimentation for 12 h, removing the supernatant, then supplement 0.9% NaCl into the flask until 600 ml and it was mixed for 10 minutes, close the mixing function and seal up and sediment. Preparation of semi-finished products: Upon sedimentation for 20 h, removing the supernatant, then supplemented 0.9% NaCl into the flask until 600 ml; mix up evenly for 30 minutes, obtaining the semi-finished hepatitis B vaccine. The batch No. of semi-finished product is S201006.

### Example 8

### Determination the Value of Semi-Finished Hepatitis B Vaccine for Absorption Completeness, Mice ED₅₀, Sedimentation Speed of Aluminum Particle and Appearance

Test Methods of Absorption Completeness:

Reagent:

Reference product: The frozen reference product of recombinant (yeast) hepatitis B vaccine comes from National Institute for the Control of Pharmaceutical and Biological Products.

Diagnostic reagent kit of hepatitis B surface antigen is purchased from Shanghai Kehua Biotechnology Co., Ltd.

Test Procedure:

The test sample was centrifuged for 5 minutes with the speed of 6500 rpm, collecting the supernatant. Measure HBsAg content in the reference product, test sample and its supernatant by the testing method of in vitro relative potency of recombinant hepatitis B vaccine (yeast). Adopt the logarithm of HBsAg content in the reference product as horizontal coordinate and adopt logarithm of corresponding absorbance as vertical coordinate for linear regression; correlation coefficient is not less than 0.99. The HBsAg content was calculated by subjecting the absorbance value of test sample and supernatant to the equation of linear regression and the absorption rate was calculated with following formula.

$$P ( \% ) = ( 1 - \frac{{C}_{s}}{{C}_{t}} ) \times 100$$

Wherein: P refers to the absorption rate of test sample, %;

Cₛ refers to HBsAg content of supernatant in the test sample in the unit of μg/ml;

Cₜ refers to HBsAg content of test sample in the unit of μg/ml.

### Detection Methods for the Median Effective Dose (ED₅₀) in Mice:

Reagent:

Diagnostic reagent kit of antigen on hepatitis B surface comes from Shanghai Kehua Biotechnology Co., Ltd.

Test animal: BALB/c mouse is purchased from Dalian Medical University.

Test Procedure:

The vaccine was diluted continuously. 14˜16 g BALB/c mice were intraperitoneally injected with 1.0 ml dilution of vaccine. 10 mice were used to injection in each dilution degree of vaccine. After raising for 4-6 weeks, the blood was collected from eyeballs of mouse about 1 ml and immune serum was prepared from the blood. Measuring the anti-HBs in the immune serum using the diagnostic reagent kit of hepatitis B surface antigen and calculating the seroconversion transfer rate according to the seroconversion quantity in each dilution degree; further calculating ED₅₀.

ED₅₀value=10^{Terminal logarithm of 50% seroconversion transfer rate}

Wherein: Terminal logarithm of 50% seroconversion transfer rate=Logarithm of dilution degree (content) of above 50% seroconversion transfer rate+distance ratio×dilution series logarithm

$$Distance ratio = \frac{( \begin{matrix}Above 50 \% seroconversation transfer rate - \\ 50 \%\end{matrix} )}{( \begin{matrix}Above 50 \% seroconversation transfer rate - \\ Less 50 \% serconversation transfer rate\end{matrix} )}$$

The detection results of absorption completeness and ED₅₀of semi-finished hepatitis B vaccine are shown in table 1. The detection results of aluminum particle sedimentation speed and appearance of semi-finished hepatitis B vaccine are shown in table 2.

TABLE 1
(The detection results of absorption completeness and ED₅₀ of
semi-finished hepatitis B vaccine)

| | | Detection of | |
|---|---|---|---|
| | | absorption | ED₅₀ |
| Process flow | Batch No. | completeness | detection |
| \textcircled{1} Aluminum adjuvant + | S201001 | 94% | 0.5 μg |
| HBsAg | S201002 | 95% | 0.5 μg |
| | S201003 | 95% | 0.6 μg |
| \textcircled{2} In-situ absorption | S201004 | 99% | 0.2 μg |
| method | S201005 | 99% | 0.3 μg |
| | S201006 | 98% | 0.2 μg |

TABLE 2
(The detection results of aluminum particle sedimentation speed
and appearance of semi-finished hepatitis B vaccine)

| | | Sedimentation | |
|---|---|---|---|
| | | speed of | |
| | | aluminum | |
| | | particle | |
| Process flow | Batch No. | (mm/min) | Appearance |
| \textcircled{1} Aluminum | S201001 | 0.43 mm/min | Milky white mixed |
| adjuvant + | | | suspension; visible particle |
| HBsAg | S201002 | 0.38 mm/min | Milky white mixed |
| | | | suspension; visible particle |
| | S201003 | 0.47 mm/min | Milky white mixed |
| | | | suspension; visible particle |
| \textcircled{2} In-situ | S201004 | 0.29 mm/min | Milky white mixed |
| absorption | | | suspension; even particle |
| method | S201005 | 0.29 mm/min | Milky white mixed |
| | | | suspension; even particle |
| | S201006 | 0.23 mm/min | Milky white mixed |
| | | | suspension; even particle |

1. A method for preparation of a hepatitis B vaccine comprising aluminum adjuvant, characterized in that aluminum adjuvant Al(OH)₃ was produced by an on-line reaction, comprises mixing PBS buffer solution, KAl(SO4)₂ solution and hepatitis B surface antigen stock solution, adding NaOH solution into the mixed solution, so that the Al(OH)₃ adjuvant is continuously produced and hepatitis B surface antigens are continuously coated and absorbed to the adjuvant simultaneously, the method comprises the following steps:
\textcircled{1} adopting injection water to prepare 3 mmol/L PBS buffer solution, 10 wt % KAl(SO₄)₂ solution, 0.5 mol/L NaOH solution and 0.9 wt % NaCl solution, and the prepared solutions were sterilized by filtration with 0.22 μm membrane filter;
\textcircled{2} adding 200 ml PBS to the reaction vessel, under the mixing condition, adding the hepatitis B surface antigen stock solution according to the content with an ultimate antigen concentration of 20 μg/ml in the semi-finished product, then adding 150 ml PBS solution and 10 wt % KAl(SO₄)₂ solution according to the aluminum content with an ultimate concentration of 0.5 mg/ml in the semi-finished product, then adding 0.5 mol/L NaOH solution, adjust pH value to 7.0, finally supplementing the 0.9 wt % NaCl solution until 600 ml and sealing the mixed solution; a semi-finished product of hepatitis B vaccine was obtained via sedimentation and washing process for the mixed solution.

2. The method according to claim 1, characterized in that the sedimentation and washing process specified in above step \textcircled{2} is as follows:
i. sedimenting the sealed mixed solution for 12-19 h, then removing the supernatant and reserve the sediment, adding the 0.9 wt % NaCl solution to the sediment until the original volume of semi-finished product, mixing the material for 30 minutes, sealing and sedimenting for the second time;
ii. repeating the above step i twice;
iii. after 4^{th} precipitation for 20 h, removing the supernatant, then adding the 0.9 wt % NaCl solution until the original volume of semi-finished product, mixing for 30 minutes and mix up evenly, obtaining the semi-finished product of hepatitis B vaccine.

3. The method according to claim 1, characterized in that the hepatitis B surface antigen comprises recombinant yeast hepatitis B surface antigen and recombinant CHO hepatitis B surface antigen.

4. The method according to claim 1, characterized in that the hepatitis B surface antigen comprises the recombinant yeast hepatitis B surface antigen.

5. The method according to claim 4, characterized in that the recombinant yeast hepatitis B surface antigen comprises recombinant hansenula polymorpha hepatitis B surface antigen.

6. The method according to claim 5, characterized in that the stock solution of recombinant hansenula polymorpha hepatitis B surface antigen is prepared by the following process:
I. collection of fermentation broth: preparing a batch strain of working seed by fourth amplification of national approved original bacteria of recombinant hansenula polymorpha hepatitis B vaccine, the batch strain seeded in 300 ml growth medium, cultured under 30˜35° C. for 24 h, and then the culture medium was transferred to 3 L growth medium further cultured under 30˜35° C. for 24 h; then the medium was transferred to 30 L growth medium cultured under 30˜35° C. for 15 h and the resulting medium ultimately transferring to 200 L growth medium and cultured under 30˜35° C. for 65-70 h in a condition of a dissolved oxygen for above 20%, a pH value at 6.8 and a air flow between 30˜200 ml/h, collecting the fermentation broth of hansenula polymorpha cell expressing the hepatitis B surface antigen;
II. preliminary purification: Grinding and crushing the fermentation broth of hansenula polymorpha cell by physical crushing method to realize a crushing rate of above 90%, removing the cell debris by centrifugation at 4000 rpm, the supernatant was performed two aqueous phase extraction by treating with NaCl/PEG6000 for 8-10 h and centrifuged with a speed of 6000 rpm, reserving the supernatant, then the supernatant was absorbed with silica gel solution for 10-16 h, and desorpted for 1 h at 60° C., then centrifuged at 4000 rpm, reserving the supernatant;
III. fine purification: purificating the preliminarily purified sample by anionic column chromatography, washing the column with 100 mmol/L tris buffer solution, monitored at absorbance of 280 nm and collecting protein peak with OD value of above 1, concentrating the protein peak for 10 times by 50K membrane, then implement centrifugal force to the belt of equivalent density area and monitored at absorbance of 280 nm, collecting a protein peak with OD value of above 1 and the protein peak was further purified by molecule sieve column chromatography, the column was eluted with biological buffer solution to perform protein separation and desalination, the elutes monitored at absorbance of 280 nm, collecting HBsAg protein peak with OD value of above 0.8 so that its purity is above 99.0%; ultimately diluting HBsAg protein peak until a content of 100-300 μg/ml, and sterilizing by filtration with 0.22 μm membrane filter, obtaining the stock solution of recombinant hansenula polymorpha hepatitis B surface antigen;
wherein the growth medium comprises the following ingredients: glycerin, magnesium sulfate, potassium chloride, sodium chloride and ammonium hydrogen phosphate, according to the mass ratio in 1 L is 5:2:1:0.1:4, it is prepared by injection water and sterilized under 121° C. for 30 minutes.

7. The method according to claim 6, characterized in that the national approved original strain of recombinant hansenula polymorpha hepatitis B vaccine enjoys a strain No. of HBsAgU35-16-9.
